(19) United States
(12) Patent Application Publication
(10) Pub. No.: US 2011/0106124 A1
(43) Pub. Date: May 5, 2011

(54) METHOD AND APPARATUS FOR ARTHROSCOPIC ROTATOR CUFF REPAIR USING TRANSOSSEOUS TUNNELS

(76) Inventor: Marc BEAUCHAMP, Montreal (CA)

(21) Appl. No.: 12/911,250

(22) Filed: Oct. 25, 2010

Related U.S. Application Data
(63) Continuation-in-part of application No. 12/785,153, filed on May 21, 2010.
(60) Provisional application No. 61/187,477, filed on Jun. 16, 2009.

(51) Int. Cl. A61B 17/34; A61B 17/06

(52) U.S. Cl. 606/170; 606/223

(57) ABSTRACT

There is provided a device for making an arthroscopic rotator cuff repair having a hooked bone-penetrating portion having a sharp tip, a shaft for transmitting force, and a gripping portion. The device may function as a suture passer, being configured to hold or pass one or more suture, and may be cannulated or have an eyelet for this purpose. There is also provided a suture passer of substantially the same shape as the device, which is configured to hold or pass one or more suture, and which may define a channel or have an eyelet for this purpose. Also disclosed herein is a kit comprising the device and suture passer. A method of making a rotator cuff is also disclosed, wherein the greater tuberosity of the humerus is perforated with the device to form a curved bone channel, and a suture is then passed through the bone channel.

### CROSS-REFERENCE TO RELATED APPLICATIONS

This application is a Continuation-In-Part Application of U.S. patent application Ser. No. 12/785,153 filed May 21, 2010, which claims the benefit of and priority to U.S. Provisional Patent Application No. 61/187,477, filed Jun. 16, 2009, both of which are incorporated herein by reference in their entirety.

### FIELD

Generally, there is provided a device and method for surgery. More specifically, there is provided a device and method for making an arthroscopic rotator cuff repair.

### BACKGROUND

Rotator cuff tear is usually an avulsion of one or more tendons from the bone (humerus). It is an extremely frequent disorder that can be secondary either to a trauma or to degeneration, such as age related degeneration. When repair is indicated, it is necessary to suture back the soft tissue (tendon) to the hard tissue (bone), thereby promoting the formation of scar tissue. The time-honoured classical method (“open approach”) is to expose both structures by an open approach that usually involves some detachment (and subsequent repair) of the deltoid muscle. Strong sutures are passed through a bone tunnel in the greater tuberosity of the humerus using a curved device (needle or suture passer). The same suture is then passed through the tendon and a knot is tied to secure it to the bone.

With the advent of arthroscopy, it became possible to perform rotator cuff repair without having to perform an “open approach” and therefore avoiding most complications related to detachment of the deltoid muscle. This technique, being also less painful and better tolerated by the patient, is becoming more and more popular. However, it is considered a much more technically demanding procedure for the surgeon than the open technique, and instead of passing sutures through bone tunnels (considered too difficult to achieve with existing instruments), suture anchors, made of metal or plastic loaded with heavy suture, are used to secure the soft tissue to the bone. These suture anchors may carry specific risks (migration, foreign body reaction, osteolysis, etc), and involve the use of a disposable screwdriver for each implantation. Many still consider the transosseous bone tunnel as the gold standard for rotator cuff repair.

Techniques for arthroscopic rotator cuff repair are known in the art.

U.S. Pat. No. 5,584,839 discloses, according to the abstract, a drill guide having a cannulated guide shaft, a sliding shaft mounted thereon, and a handle for pulling back the sliding shaft. The sliding shaft comprises a hook for holding bone between the hook and the cannulated guide shaft. Pulling back on the sliding shaft holds the bone while a drill is passed through the drill guide to form a channel. The tissue repair is completed by passing suture ends through the tunnel. However the bone tunnel is relatively short and shallow, which may result in increased fragility. Further, the device must be placed onto the deltoid muscle, raising the possibility of injury.

U.S. Pat. No. 5,681,333 discloses, according to the abstract, a similar straight cannulated drill guide having a sliding aiming arm for aligning a multifunctional drillhook. The aiming arm is lockable with respect to the drill guide and has a radiused (curved) end for clearing the greater tuberosity. The drillhook marks the drillhook exit point and may have a spiked end for stabilizing the drill guide. After drilling the tunnel, a curved perforating suture hook (which terminates in 70 degrees of arc), is used to pass the suture through the rotator cuff (tendon) and into a position for the drillhook to retrieve and pull it through the tunnel. However, here too the bone tunnel is straight and relatively shallow and may be more fragile. Placement of the device during the operation may damage the deltoid muscle.

U.S. Patent Publication 20070208356 discloses, according to the abstract, a surgical apparatus for use in arthroscopic procedures comprising drill guides for drilling first and second perpendicular intersecting holes. The apparatus may also comprise a suture retriever for retrieving from the second tunnel a suture which has been fed into the first tunnel. However, this device requires the surgeon to form two holes which meet at an exact point and requires the suture to be fed around a corner of about 90 degrees, which is known to be problematic and to make retrieval more difficult.

U.S. Patent Publication 20070088362 discloses an instrument for guiding and positioning implants within the body comprising a guide channel connected with a hook. The guide channel is disposed in a body member, the longitudinal axis of which is aligned with the hook. During use, the hook may be placed on or around bone and a drill system may be used to create a passage through bone which terminates at a point near the end of the hook. The hook may be hollow for passage of a suture loop which may be grabbed and pulled through the bone tunnel by a grabber. However, here too the bone tunnel is straight and the placement of the instrument onto bone could damage muscles.

These previous approaches are also characterized by instruments with moving parts which may be expensive and which may pose sterilization problems. It would be advantageous to have a simple apparatus for making arthroscopic rotator cuff repairs which would allow strong tunnels to be formed in a shape which would facilitate the passing of sutures during surgery. It would also be advantageous for such a device to pose less of a risk for tissue damage.

### SUMMARY

In one aspect, there is provided a device for use in making an arthroscopic rotator cuff repair comprising:
a hooked bone-penetrating portion for creating a curved bone tunnel,
a shaft for transmitting force to said hooked bone-penetrating portion, and
a gripping portion for manipulating said device,
wherein said hooked bone-penetrating portion comprising a sharp tip for penetrating bone and further comprises 140 to 200 degrees of arc.

In another aspect, there is provided suture passer for passing a suture, wire, loop, or flexible metal jaw hook through a curved bone tunnel, said suture passing comprising:
a hooked portion for passing through said bone tunnel,
a shaft coupled to said hooked portion, and
a gripping portion for manipulating said suture passer,
wherein said suture passer is configured to hold a suture, wire, loop, or flexible metal jaw hook, said hooked portion of said suture passer comprising 140 to 210 degrees of arc.

In another aspect, there is provided a kit comprising the above-described device together with a suture passer for passing a suture, wire, loop, or flexible metal jaw hook through a bone tunnel formed by the above-described device, said suture passing comprising:
a hooked portion for passing through said bone tunnel,
a shaft coupled to said hooked portion, and
a gripping portion for manipulating said suture passer,
wherein said suture passer is configured to hold a suture, wire, loop, or flexible metal jaw hook, said hooked portion of said suture passer comprising 140 to 210 degrees of arc.

In another aspect, there is provided a method of making an arthroscopic rotator cuff repair comprising the steps of:
penetrating the greater tuberosity of a humerus with the above-described device to form a curved bone channel;
passing a suture, wire, loop, or flexible metal jaw hook through said curved bone tunnel; and
securing a tendon to said greater tuberosity with said suture, wire, loop, or flexible metal jaw hook to make said rotator cuff repair.

Other aspects and embodiments will become clear to a skilled person upon reading the following description in conjunction with the Figures.

### BRIEF DESCRIPTION OF THE DRAWINGS

Embodiments will be described, by way of example, with reference to the Figures, wherein:

FIG. 1A depicts a bone-penetrating awl, according to one embodiment;

FIG. 1B depicts a bone-penetrating awl, according to another embodiment;

FIG. 1C depicts a bone-penetrating awl, according to a further embodiment;

FIG. 1D depicts a bone-penetrating awl, according to another embodiment;

FIG. 1E depicts a bone-penetrating awl, according to another embodiment;

FIG. 2A depicts a suture passer, according to one embodiment;

FIG. 2B depicts a suture passer, according to another embodiment;

FIG. 2C depicts a suture passer, according to a further embodiment;

FIG. 2D depicts a suture passer, according to another embodiment;

FIG. 2E depicts a suture passer, according to another embodiment;

FIG. 2F depicts a tip of a suture passer depicted as F in FIGS. 2B, 2D, and 2F according to one embodiment;

FIG. 2G depicts a tip of a suture passer depicted as G in FIG. 2A, according to another embodiment;

FIG. 2H depicts a tip of a suture passer depicted as H in FIG. 2C, according to another embodiment;

FIG. 3A depicts a step of a surgical method, in accordance with one embodiment;

FIG. 3B depicts a step of a surgical method, in accordance with one embodiment;

FIG. 3C depicts a step of a surgical method, in accordance with one embodiment;

FIG. 3D depicts a step of a surgical method, in accordance with one embodiment;

FIG. 3E depicts a step of a surgical method, in accordance with one embodiment; and

FIG. 3F depicts a step of a surgical method, in accordance with one embodiment.

### DESCRIPTION

Generally, there is provided, in one aspect, a device for making an arthroscopic rotator cuff repair having a hooked bone-penetrating portion with a sharp tip, a shaft for transmitting force, and a gripping portion. In some embodiments, the device may function as a suture passer, being configured to hold or pass one or more suture. It may be cannulated or define an eyelet for this purpose. In another aspect, there is provided a suture passer of substantially the same shape as the device, which is configure to hold or pass one or more suture. In some embodiments, the suture passer defines a channel or an eyelet for this purpose. In another aspect, there is provided a kit comprising the device and the suture passer. In a further aspect, a method of making a rotator cuff is also provided, wherein the greater tuberosity of the humerus is perforated with the device to form a curved bone channel, and a suture is then passed through the bone channel.

In one aspect, there is provided a device for use in making an arthroscopic rotator cuff repair comprising:
a hooked bone-penetrating portion for creating a curved bone tunnel,
a shaft for transmitting force to said hooked bone-penetrating portion, and
a gripping portion for manipulating said device,
wherein said hooked bone-penetrating portion comprising a sharp tip for penetrating bone and further comprises 140 to 200 degrees of arc.

The hooked bone-penetrating portion may comprise 180 to 200 degrees of arc. In another one embodiment, the hooked bone-penetrating portion comprises a radius of curvature of about 12 mm. The device may be configured to hold a suture, wire, loop, or flexible metal jaw hook. The device may define a channel configured to hold said suture, wire, loop, or flexible metal jaw hook, said channel extending from a first opening defined in said hooked bone-penetrating portion to a second opening defined in said shaft. The device may define an eyelet configured to hold said suture, wire, loop, or flexible metal jaw hook, said eyelet being defined in said hooked bone-penetrating portion. The shaft and said hooked bone-penetrating portion may be angled with respect to one another to recapitulate an angle between a curved surgical needle and a needle holder.

In another aspect, there is provided a suture passer for passing a suture, wire, loop, or flexible metal jaw hook through a curved bone tunnel, said suture passing comprising:
a hooked portion for passing through said bone tunnel,
a shaft coupled to said hooked portion, and
a gripping portion for manipulating said suture passer,
wherein said suture passer is configured to hold a suture, wire, loop, or flexible metal jaw hook, said hooked portion of said suture passer comprising 140 to 210 degrees of arc.

The curved bone tunnel may be formed by the above-described device. The suture passer may be of substantially the same shape and of a cross-sectional thickness which is less than or equal to said above-described device. The hooked portion of said suture passer may comprise 190 to 210 degrees of arc. The hooked bone-penetrating portion of said device and said hooked portion of said suture passer may each have a radius of curvature of about 12 mm. The suture passer may define a channel configured to hold said suture, wire, loop, or flexible metal jaw hook, said channel extending from a first opening defined in said hooked portion to a second opening defined in said shaft. The suture passer may define an eyelet configured to hold said suture, wire, loop, or flexible metal jaw hook, said eyelet being defined in said hooked bone-penetrating portion. The hooked portion of said suture passer may be longer than said hooked bond-penetrating portion of said device.

In another aspect, there is provided a kit comprising the above-described device together with a suture passer for passing a suture, wire, loop, or flexible metal jaw hook through a bone tunnel formed by the above-described device, said suture passing comprising:
a hooked portion for passing through said bone tunnel,
a shaft coupled to said hooked portion, and
a gripping portion for manipulating said suture passer,
wherein said suture passer is configured to hold a suture, wire, loop, or flexible metal jaw hook, said hooked portion of said suture passer comprising 140 to 210 degrees of arc.

The hooked bone-penetrating portion of said device and said hooked portion of said suture passer may each have a radius of curvature of about 12 mm. The hooked portion of said suture passer may comprise 190 to 210 degrees of arc. The suture passer may define a channel configured to hold said suture, wire, loop, or flexible metal jaw hook, said channel extending from a first opening defined in said hooked portion to a second opening defined in said shaft. The suture passer may define an eyelet configured to hold said suture, wire, loop, or flexible metal jaw hook, said eyelet being defined in said hooked bone-penetrating portion. The hooked portion of said suture passer may be longer than said hooked bone-penetrating portion of said device.

In another aspect, there is provided a method of making an arthroscopic rotator cuff repair comprising the steps of:
penetrating the greater tuberosity of a humerus with the above-described device to form a curved bone channel;
passing a suture, wire, loop, or flexible metal jaw hook through said curved bone tunnel; and
securing a tendon to said greater tuberosity with said suture, wire, loop, or flexible metal jaw hook to make said rotator cuff repair.

The hooked bone-penetrating portion may comprise 180 to 200 degrees of arc. The hooked bone-penetrating portion may comprise a radius of curvature of about 12 mm. The step of passing may be accomplished with the above-described device, said device being configured to hold a suture, wire, loop, or flexible metal jaw hook. The device may define a channel configured to hold said suture, wire, loop, or flexible metal jaw hook, said channel extending from a first opening defined in said hooked bone-penetrating portion to a second opening defined in said shaft. The device may define an eyelet configured to hold said suture, wire, loop, or flexible metal jaw hook, said eyelet being defined in said hooked bone-penetrating portion.

The passing step may accomplished with a suture passer comprising:
a hooked portion for passing through said bone tunnel,
a shaft coupled to said hooked portion, and
a gripping portion for manipulating said suture passer,
wherein said suture passer is configured to hold a suture, wire, loop, or flexible metal jaw hook, said hooked portion of said suture passer comprising 140 to 210 degrees of arc.

The hooked portion of said suture passer may comprise 190 to 210 degrees of arc. The hooked bone-penetrating portion of said device and said hooked portion of said suture passer may each have a radius of curvature of about 12 mm. The suture passer may define a channel configured to hold said suture, wire, loop, or flexible metal jaw hook, said channel extending from a first opening defined in said hooked portion to a second opening defined in said shaft. The suture passer may define an eyelet configured to hold said suture, wire, loop, or flexible metal jaw hook, said eyelet being defined in said hooked bone-penetrating portion.

Penetrating may comprise:
penetrating said greater tuberosity at a first location to form a first partial curved bone tunnel, and
penetrating said greater tuberosity at a second location to form a second partial curved bone tunnel in communication with said first partial bone tunnel, said first and second partial curved bone tunnels together having a substantially continuous curvature.

Prior to penetrating, an initial perforation may be made into said greater tuberosity with a straight instrument.

Device

In one aspect, there is provided a device for making an arthroscopic rotator cuff repair.

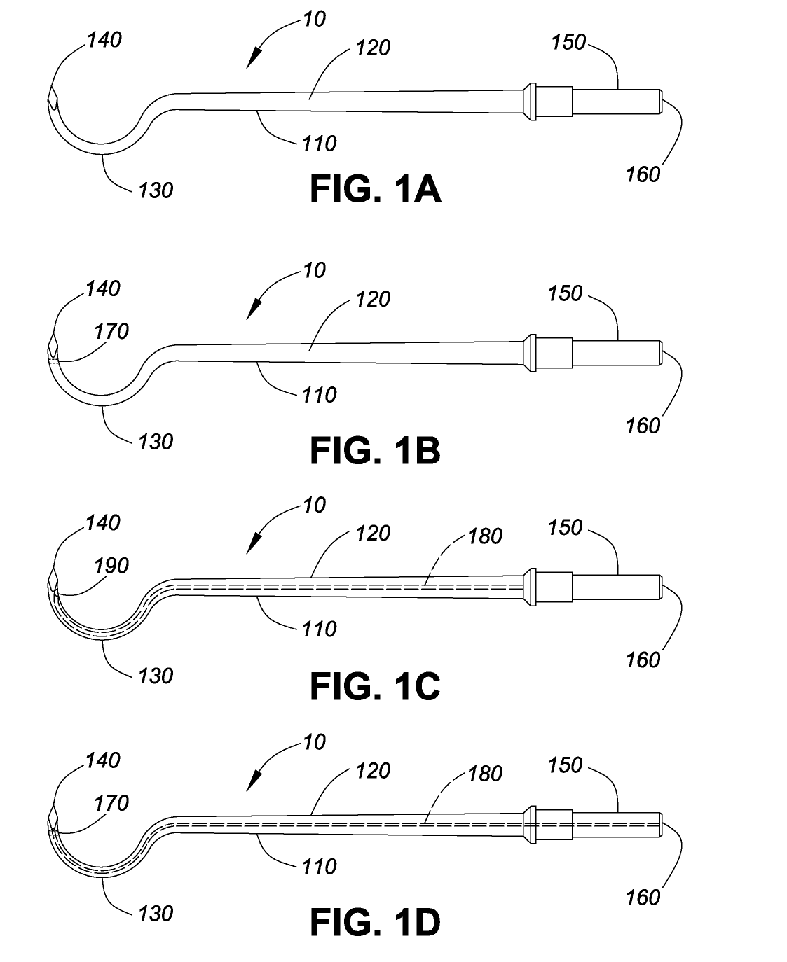

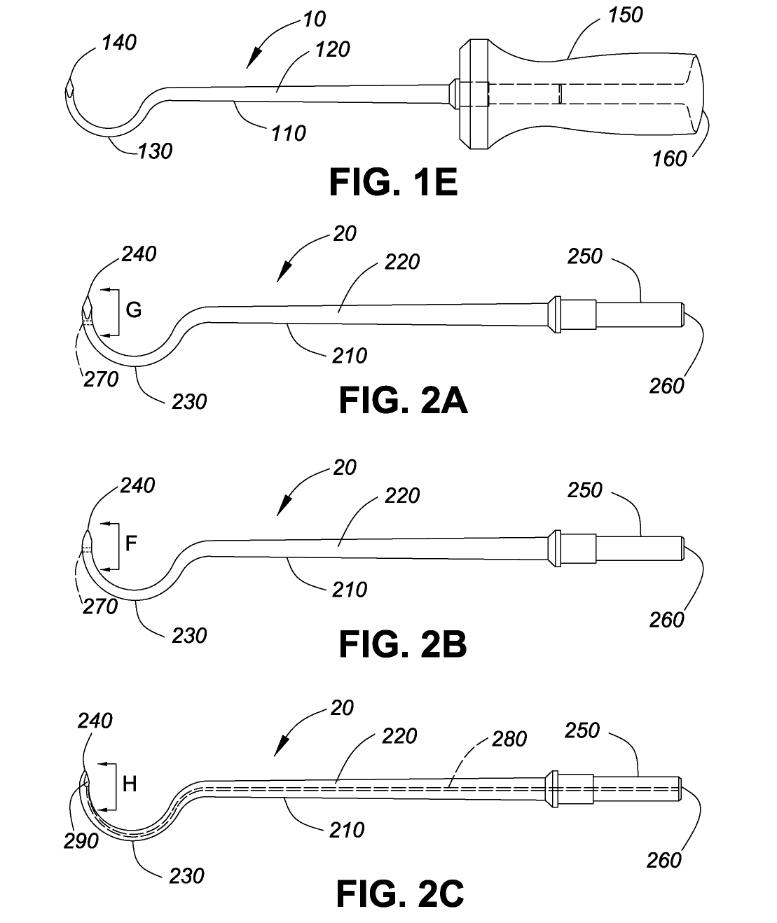

In one embodiment, the device comprises a bone-penetrating awl. Referring to FIG. 1A, the device 10 comprises a gripping portion for manipulating and a shaft 110 having a substantially straight proximal portion 120, the shaft being for transmitting bone-penetrating force to a bone-penetrating hooked portion 130, which is for creating a bone tunnel. The bone-penetrating hooked portion has a tip 140 for penetrating bone. Preferably, the device 10 is a handheld instrument, with the gripping portion comprising a handle 150, which may be formed thereon. Being preferably handheld, the apparatus may be inserted and manipulated manually, without the use of power tools, so force can be controlled by the surgeon at all times, which may decrease the risk of iatrogenic bone damage

The hooked bone-penetrating portion has a selected radius of curvature of 9 to 15 mm, more preferably 10 to 14 mm, more preferably 11 to 13 mm most preferably about 12 mm. Numerical values expressed herein with the term “about” are intended to encompass the recited value plus or minus 5%. The hooked portion comprises 140 to 200 degrees, preferably 150 to 200 degrees, more preferably 160 to 200 degrees, more preferably 170 to 200 degrees, more preferably 180 to 200, or more preferably still 185 to 195 degrees of arc. Preferably, it comprises about 189 degrees of arc. In one embodiment, the hooked portion comprises 189.5 degrees of arc. It may have a curvature similar to a curved needle. The size of the hook may depend on the age and size of the patient. The curvature of the hooked portion allows for an all-arthroscopic technique, which is minimally invasive and may result in fewer complications or be less painful than an “open” approach.

The tip 140 is sharp in order to penetrate bone. The tip 140 comprises a point. Preferably, it is substantially diamond shaped. Even more preferable, the diamond shape may comprise three, four, or five sides. The tip may terminate 0 to 4 mm or, preferably, 1 to 3 mm beyond 180 degrees of arc. Most preferably, it terminates about 2 mm beyond 180 degrees of arc. The tip may be curved in conformity with the curve of the hooked portion (as in FIGS. 1B to 1E), or, preferably, it may be substantially straight (see, for example, FIG. 1A). Most preferably, as in FIG. 1A, it may be straight and project tangentially from the point at which is it joined to the curved portion of the hook.

The hooked bone-penetrating portion 130 of the shaft may taper towards the tip 140. The substantially straight portion 120 may taper towards the hooked bone-penetrating portion 130. A skilled person would appreciate that the hooked portion must have a minimum diameter to withstand the forces generated during percussion. The substantially straight portion may taper from 5.6 to 6.25 mm to 1.8 to 2.5 mm. Preferably, the substantially straight portion tapers from about 6.25 mm to about 2.5 mm.

It is preferred that the device may have a substantially round, square, rectangular, diamond-shaped or oval in cross-sectional shape. Preferably the awl has a round cross-sectional shape in order to facilitate turning of the instrument inside the bone tunnel, for example, during formation in order to correct the direction of the tip, if required.

In some embodiments, the shaft 110 and hooked portion 130 may be angled with respect to one another. In such embodiments, they may be angled 30 to 60, more preferably 40 to 50, or still more preferably about 45 degrees with respect to one another. The obliquity may recapitulate the angle between a curved needle and a needle holder as used during surgery. By “recapitulate” is meant that the angle approximates, mimics, or is similar to the angle between a curved needle and a needle holder as used during surgery. The angles need not be precisely identical.

In one embodiment, the device also functions as a suture passer and may be configured to hold a suture.

By “hold” (as used throughout) is meant that the device would retain a suture securely enough to allow it to be passed through a bone channel. In some embodiments, “hold” is intended to encompass receiving or passing a suture through a bone tunnel. In some embodiments, “hold” encompasses a suture being received or passed (for example, threaded) through a device which itself has already been passed through or engaged with a bone tunnel.

By “configured to hold” is meant that the device is capable of receiving, holding, or passing a suture. In some embodiments, it may encompass the device having one or more aperture, eyelet, opening, groove, hook, recess, or channel through which a suture may be threaded. The means of holding may also encompass a means for releasably securing a suture to part of the device. For example, adhesives, clips, or clasps may be used to secure a suture to part of the device.

Although “suture” is referred to throughout, a skilled person would appreciate that the device, when used as a suture passer, would, in some embodiments, be capable of passing one or more sutures, wires, loops, flexible metal jaw hooks, or any appropriate means of securing a tendon to make a rotator cuff repair. In some embodiments, a metal wire having, for example, a looped or diamond-shaped end may be threaded through the device (e.g. through an aperture, eyelet, groove, channel, etc.) and used to pull or thread one or more sutures back through the device. In some embodiments, the device may be engaged with a bone channel while this is being carried out.

Referring to FIG. 1B, the hooked portion 130 of awl 10 may define an eyelet 170 extending through the width of a portion of the hooked portion, which is configured to hold a suture. Preferably, the eyelet 170 is located just below the tip 140. The eyelet may be any shape suited to receiving a suture. Preferably, it is shaped and sized to receive 2 or 3 heavy sutures, a tape, or a wire-loop. The eyelet may be round. Preferably, the eyelet is oval. Even more preferable, the eyelet is sized about 1.25 mm×1.95 mm.

When used as a passer, the device may be configured to hold one or more suture or metal wire during normal use. Referring to FIG. 1C, the device may define a channel 180 or cannula configured to hold one or more suture or metal wires. Preferably the channel 180 terminates at an opening 190 defined near the tip 140. Opening 190 may be round, but preferably it is oval. The second end of channel 180 may be at an opening defined by the proximal end 160 of the shaft 110. In other embodiments, the second end of channel may be at an opening situated elsewhere along the shaft 110 or hooked portion 130. The channel may be of any size suited to receiving one or more sutures or metal wires which provides an awl of sufficient strength for normal use.

Like the opening 190, the channel 180 is preferably sized to receive and pass two or three heavy sutures, a tape, or a wire-loop.

Other means for retaining one or more sutures or metal wires in or on the awl during use are also fully contemplated herein. For instance, one or more sutures or metal wires may be held in an accommodating groove extending along at least a portion of the shaft 110 or along the entire length of the shaft 110. The one or more sutures or metal wires may also be threaded through eyelets spaced along the shaft 110 of the awl. These means of retaining may also include fasteners for reversibly securing the one or more sutures or metal wires to the awl during normal use. These means of securing would preferably involve threading the suture or metal wire through an opening or eyelet situated near the tip 140.

In another embodiment exemplified in FIG. 1D, eyelet 170 is in communication with a channel 180 defined by shaft 110 and extending therethrough to an opening defined in the proximal end 160, the channel being for receiving and passing a suture. Preferably, the channel should be sized to receive two or three heavy sutures. The channel may extend through the length of the shaft 110, as shown in FIG. 1 D. However, it may also extend through a shorter portion of the shaft to an opening defined elsewhere on the shaft.

In the embodiments in which the awl also functions as a suture passer, a skilled person would appreciate that the awl comprising an eyelet or channel would also need to be of sufficient hardness to permit perforation of bone, for example, by percussion.

The shaft 110 or handle 150 preferably comprises a flat surface to allow percussion, for example, with a mallet. For example, the proximal end 160 of the shaft may be flat to allow percussion. In a preferred embodiment, depicted in FIG. 1E, the handle 150 may be of greater diameter than the shaft 110 and may be shaped and sized to facilitate gripping. It may be formed of a material, such as silicone, rubber, plastic, or metal to facilitate gripping and may also be textured for the same purpose. Preferably, it is formed of silicone.

The awl may be formed of stainless steel. Preferably, it is formed of stainless steel 455. Preferably, it is in H900 condition. More preferably, it has a hardness minimum of 49 HRC (Rockwell scale).

The awl may be supplied in one piece, or may be assembled from parts, which may correspond to one or more of the gripping portion, shaft, hooked portion, or tip. Due to the simplicity of construction, the awl or a part thereof may be readily sterilized, reused, and, therefore, is more “environmentally friendly”. However, the awl or a part thereof may be disposable.

Suture Passer

In another aspect, there is provided a suture passer for making an arthroscopic rotator cuff repair.

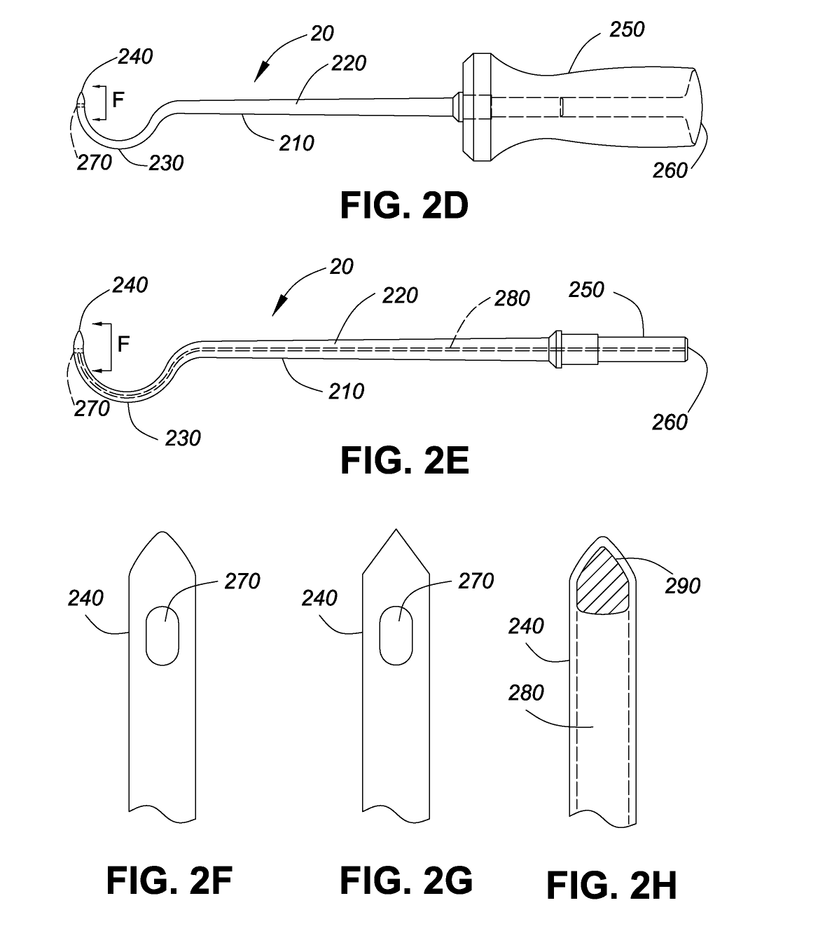

In one embodiment, the suture passer may be for use with the above-described device. Referring to FIG. 2A, the suture passer 20 is provided to pass a suture through a bone tunnel formed by the above-described device. The suture passer 20 comprises a gripping portion 250 for manipulating the suture passer and a shaft 210 having a substantially straight proximal portion 220 coupled to a hooked portion 230 having a tip 240.

Although “suture” is referred to throughout, a skilled person would appreciate that the device, when used as a suture passer, would, in some embodiments, be capable of passing one or more sutures, wires, loops, flexible metal jaw hooks, or any appropriate means of securing a tendon to make a rotator cuff repair. In some embodiments, a metal wire having, for example, a looped or diamond-shaped end may be threaded through the device (e.g. through an aperture, eyelet, groove, channel, etc.) and used to pull or thread one or more sutures back through the device.

The suture passer is preferably of substantially the same shape and cross-sectional size as the device described above. “Substantially the same shape”, as referred to herein, indicates that the suture passer is shaped so that its hooked portion is able to fit into (and its tip pass through) a bone channel formed by the awl described above. In some embodiments, the suture passer may also be of a smaller cross-sectional size than the awl.

The hooked portion has a selected radius of curvature of 9 to 15 mm, more preferably 10 to 14 mm, more preferably 11 to 13 mm most preferably about 12 mm. The hooked portion comprises 150 to 210 degrees, preferably 160 to 210 degrees, more preferably 170 to 210 degrees, more preferably still 180 to 210 degrees, even more preferably 190 to 210 degrees, or even more preferably still 195 to 205 degrees of arc. Preferably, it comprises about 199 degrees of arc. In one embodiment, the hooked portion comprises about 199 degrees of arc. It may have a curvature similar to a curved needle. The size of the hooked portion may depend on the age and size of the patient.

Preferably, the suture passer has a substantially round cross-sectional shape. However, it may also have a square, diamond-shaped or oval cross-sectional shape. Whatever the cross-sectional shape, a skilled person would appreciate that the suture passer is sized to fit into the curved bone tunnel formed by the awl.

In some embodiments, the shaft 210 and hooked portion 230 may be angled with respect to one another. In such embodiments, they may be angled 30 to 60, more preferably 40 to 50, or still more preferably about 45 degrees with respect to one another. The angle recapitulate the angle between a curved needle and a needle holder as used during surgery.

As seen in FIG. 2B and 2F, the tip 240 of suture passer 20 is preferably blunt. More preferably, it is a rounded point or round. However, the tip 240 may also be pointed or sharp, as may be seen in FIGS. 2A or 2G.

It is preferred that the hooked portion 230 of the suture passer be longer than the hooked bone-penetrating portion 130 of the awl to facilitate passage of the suture through the curved bone tunnel. Tip 240 may extend 0 to 6 mm beyond 180 degrees of arc. Preferably, tip 240 extends 2 to 6 mm beyond 180 degrees of arc. Even more preferable, tip 240 terminates about 4 mm beyond 180 degrees of arc.

The suture passer is configured to hold one or more sutures or metal wire during normal use.

By “hold” (as used throughout) is meant that the suture passer would retain a suture securely enough to allow it to be passed through a bone channel. In some embodiments, “hold” is intended to encompass receiving or passing a suture through a bone tunnel. In some embodiments, “hold” encompasses a suture being received or passed through a suture which itself has already been passed through or engaged with a bone tunnel.

By “configured to hold” is meant that the suture passer is capable of receiving, holding, or passing a suture. In some embodiments, it may encompass the device having one or more aperture, eyelet, opening, groove, hook, recess, or channel through which a suture may be threaded. The means of holding may also encompass a means for releasably securing a suture to part of the device. For example, adhesives, clips, or clasps may be used to secure a suture to part of the device.

Referring to FIG. 2C, the suture passer may define a channel 280 or cannula configured to receiving or hold one or more suture or metal wires. Preferably the channel 280 terminates at an opening 290 defined at or near the tip. FIG. 2H depicts one preferred example of a channel 280 exiting at an opening 290 defined by tip 240. Opening 290 may be round, but preferably it is oval. The second end of channel 280 may be at an opening defined by the proximal end 260 of the shaft 210. In other embodiments, the second end of channel 280 may be at an opening situated elsewhere along the shaft 210 or hooked portion 230. The channel may be of any size suited to receiving one or more sutures or metal wires which provides a suture passer of sufficient strength for normal use.

Like the opening 290, the channel 280 is preferably sized to receive and pass two or three heavy sutures, a tape, or a wire-loop.

Other means for holding or passing one or more suture or metal wire in or on the suture passer during use are also fully contemplated herein. For instance, one or more sutures or metal wires may be held in an accommodating groove extending along at least a portion of the shaft 210 or along the entire length of the shaft 210. The one or more sutures or metal wires may also be threaded through eyelets spaced along the shaft 210 of the suture passer. These means of retaining may also include fasteners for reversibly securing the one or more suture or metal wire to the suture passer during normal use. These means of securing may involve threading the suture or metal wire through an opening or eyelet situated at or near the tip 240.

Referring to FIG. 2B, hooked portion 230 may define an eyelet 270 configured to receive or hold one or more suture or metal wire. Preferably, the eyelet 270 is situated towards the tip 240. Preferably, as depicted, for example, in FIG. 2F and 2G, the eyelet 270 is oval, though it may also be round. Even more preferably, it is oval and sized about 1.25 mm×1.95 mm. However, an eyelet of any shape or size suited to receiving and passing one or more sutures or metal wires is fully contemplated herein.

As depicted in FIG. 2E, the eyelet 270 may be in communication with a channel 280 extending through at least part of the hooked portion 230 so that a suture may be passed therethrough. Preferably, the channel should be sized to receive two or three heavy sutures. The channel may pass through the length of the shaft 210 and terminate at an opening in the proximal end 260. However, it may also pass through a shorter portion of the shaft and to an opening defined elsewhere on the shaft.

The hooked end 230 of the shaft may taper towards the tip 240. The substantially straight portion 220 may taper towards the hooked end 230. The substantially straight portion may taper from 3 to 6.5 mm to 1.2 to 2.5 mm. Preferably, the substantially straight portion tapers from 6.25 mm to 2.5 mm.

The shaft 210 or handle 250 comprises a flat surface to allow percussion, for example, with a mallet. Preferably, the proximal end of the shaft 260 may be flat to allow percussion. In a preferred embodiment, depicted in FIG. 2D, the handle 250 may be of greater diameter than the shaft 210 and may be shaped and sized to facilitate gripping. It may be formed of a material, such as silicone, rubber, metal, or plastic, to facilitate gripping and may also be textured for the same purpose. Preferably, it is formed of silicone.

The suture passer may be formed of stainless steel, such as stainless steel 455 in H900 condition. It may have a hardness minimum of 49 HRC (Rockwell scale).

The suture passer may be supplied in one piece, or may be assembled from parts, which may correspond to one or more of the gripping portion, shaft, hooked portion, or tip. Owing to the simplicity of construction, the suture passer or a part thereof may be readily sterilized, reused, and, therefore, may be more “environmentally friendly”. However, in another embodiment, the suture passer or a part thereof may be disposable and/or formed of aluminum or plastic.

Kit Comprising an Awl and a Suture Passer

In another aspect, there is provided a kit comprising the awl as described above and the suture passer as described above. The device comprises a bone-penetrating awl. Referring to FIG. 1A, the device 10 comprises a gripping portion for manipulating and a shaft 110 having a substantially straight proximal portion 120, the shaft being for transmitting bone-penetrating force to a bone-penetrating hooked portion 130, which is for creating a bone tunnel. The bone-penetrating hooked portion has a tip 140 for penetrating bone. Preferably, the device 10 is a handheld instrument, with the gripping portion comprising a handle 150, which may be formed thereon. Being preferably handheld, the apparatus may be inserted and manipulated manually, without the use of power tools, so force can be controlled by the surgeon at all times, which may decrease the risk of iatrogenic bone damage

The hooked bone-penetrating portion has a selected radius of curvature of 9 to 15 mm, more preferably 10 to 14 mm, more preferably 11 to 13 mm most preferably about 12 mm. Numerical values expressed herein with the term “about” are intended to encompass the recited value plus or minus 5%. The hooked portion comprises 140 to 200 degrees, preferably 150 to 200 degrees, more preferably 160 to 200 degrees, more preferably 170 to 200 degrees, more preferably 180 to 200, or more preferably still 185 to 195 degrees of arc. Preferably, it comprises about 189 degrees of arc. In one embodiment, the hooked portion comprises 189.5 degrees of arc. It may have a curvature similar to a curved needle. The size of the hook may depend on the age and size of the patient. The curvature of the hooked portion allows for an all-arthroscopic technique, which is minimally invasive and may result in fewer complications or be less painful than an “open” approach.

The tip 140 is sharp in order to penetrate bone. The tip 140 comprises a point. Preferably, it is substantially diamond shaped. Even more preferable, the diamond shape may comprise three, four, or five sides. The tip may terminate 0 to 4 mm or, preferably, 1 to 3 mm beyond 180 degrees of arc. Most preferably, it terminates about 2 mm beyond 180 degrees of arc. The tip may be curved in conformity with the curve of the hooked portion (as in FIGS. 1B to 1E), or, preferably, it may be substantially straight (see, for example, FIG. 1A). Most preferably, as in FIG. 1A, it may be straight and project tangentially from the point at which is it joined to the curved portion of the hook.

The hooked bone-penetrating portion 130 of the shaft may taper towards the tip 140. The substantially straight portion 120 may taper towards the hooked bone-penetrating portion 130. A skilled person would appreciate that the hooked portion must have a minimum diameter to withstand the forces generated during percussion. The substantially straight portion may taper from 5.6 to 6.25 mm to 1.8 to 2.5 mm. Preferably, the substantially straight portion tapers from about 6.25 mm to about 2.5 mm.

It is preferred that the device may have a substantially round, square, rectangular, diamond-shaped or oval in cross-sectional shape. Preferably the awl has a round cross-sectional shape in order to facilitate turning of the instrument inside the bone tunnel, for example, during formation in order to correct the direction of the tip, if required.

In some embodiments, the shaft 110 and hooked portion 130 may be angled with respect to one another. In such embodiments, they may be angled 30 to 60, more preferably 40 to 50, or still more preferably about 45 degrees with respect to one another. The obliquity may recapitulate the angle between a curved needle and a needle holder as used during surgery.

By “recapitulate” is meant that the angle approximates, mimics, or is similar to the angle between a curved needle and a needle holder as used during surgery. The angles need not be precisely identical.

In one embodiment, the device also functions as a suture passer and may be configured to hold a suture.

By “hold” (as used throughout) is meant that the device would retain a suture securely enough to allow it to be passed through a bone channel. In some embodiments, “hold” is intended to encompass receiving or passing a suture through a bone tunnel. In some embodiments, “hold” encompasses a suture being received or passed (for example, threaded) through a device which itself has already been passed through or engaged with a bone tunnel.

By “configured to hold” is meant that the device is capable of receiving, holding, or passing a suture. In some embodiments, it may encompass the device having one or more aperture, eyelet, opening, groove, hook, recess, or channel through which a suture may be threaded. The means of holding may also encompass a means for releasably securing a suture to part of the device. For example, adhesives, clips, or clasps may be used to secure a suture to part of the device.

Although “suture” is referred to throughout, a skilled person would appreciate that the device, when used as a suture passer, would, in some embodiments, be capable of passing one or more sutures, wires, loops, flexible metal jaw hooks, or any appropriate means of securing a tendon to make a rotator cuff repair. In some embodiments, a metal wire having, for example, a looped or diamond-shaped end may be threaded through the device (e.g. through an aperture, eyelet, groove, channel, etc.) and used to pull or thread one or more sutures back through the device. In some embodiments, the device may be engaged with a bone channel while this is being carried out.

Referring to FIG. 1B, the hooked portion 130 of awl 10 may define an eyelet 170 extending through the width of a portion of the hooked portion, which is configured to hold a suture. Preferably, the eyelet 170 is located just below the tip 140. The eyelet may be any shape suited to receiving a suture. Preferably, it is shaped and sized to receive 2 or 3 heavy sutures, a tape, or a wire-loop. The eyelet may be round. Preferably, the eyelet is oval. Even more preferable, the eyelet is sized about 1.25 mm×1.95 mm.

When used as a passer, the device may be configured to hold one or more suture or metal wire during normal use.

Referring to FIG. 1C, the device may define a channel 180 or cannula configured to hold one or more suture or metal wires. Preferably the channel 180 terminates at an opening 190 defined near the tip 140. Opening 190 may be round, but preferably it is oval. The second end of channel 180 may be at an opening defined by the proximal end 160 of the shaft 110. In other embodiments, the second end of channel may be at an opening situated elsewhere along the shaft 110 or hooked portion 130. The channel may be of any size suited to receiving one or more sutures or metal wires which provides an awl of sufficient strength for normal use.

Like the opening 190, the channel 180 is preferably sized to receive and pass two or three heavy sutures, a tape, or a wire-loop.

Other means for retaining one or more sutures or metal wires in or on the awl during use are also fully contemplated herein. For instance, one or more sutures or metal wires may be held in an accommodating groove extending along at least a portion of the shaft 110 or along the entire length of the shaft 110. The one or more sutures or metal wires may also be threaded through eyelets spaced along the shaft 110 of the awl. These means of retaining may also include fasteners for reversibly securing the one or more sutures or metal wires to the awl during normal use. These means of securing would preferably involve threading the suture or metal wire through an opening or eyelet situated near the tip 140.

In another embodiment exemplified in FIG. 1D, eyelet 170 is in communication with a channel 180 defined by shaft 110 and extending therethrough to an opening defined in the proximal end 160, the channel being for receiving and passing a suture. Preferably, the channel should be sized to receive two or three heavy sutures. The channel may extend through the length of the shaft 110, as shown in FIG. 1D. However, it may also extend through a shorter portion of the shaft to an opening defined elsewhere on the shaft.

In the embodiments in which the awl also functions as a suture passer, a skilled person would appreciate that the awl comprising an eyelet or channel would also need to be of sufficient hardness to permit perforation of bone, for example, by percussion.

The shaft 110 or handle 150 preferably comprises a flat surface to allow percussion, for example, with a mallet. For example, the proximal end 160 of the shaft may be flat to allow percussion. In a preferred embodiment, depicted in FIG. 1E, the handle 150 may be of greater diameter than the shaft 110 and may be shaped and sized to facilitate gripping. It may be formed of a material, such as silicone, rubber, plastic, or metal to facilitate gripping and may also be textured for the same purpose. Preferably, it is formed of silicone.

The awl may be formed of stainless steel. Preferably, it is formed of stainless steel 455. Preferably, it is in H900 condition. More preferably, it has a hardness minimum of 49 HRC (Rockwell scale).

The awl may be supplied in one piece, or may be assembled from parts, which may correspond to one or more of the gripping portion, shaft, hooked portion, or tip. Due to the simplicity of construction, the awl or a part thereof may be readily sterilized, reused, and, therefore, is more “environmentally friendly”. However, the awl or a part thereof may be disposable.

The suture passer may be for use with the above-described device. Referring to FIG. 2A, the suture passer 20 is provided to pass a suture through a bone tunnel formed by the above-described device. The suture passer 20 comprises a gripping portion 250 for manipulating the suture passer and a shaft 210 having a substantially straight proximal portion 220 coupled to a hooked portion 230 having a tip 240.

Although “suture” is referred to throughout, a skilled person would appreciate that the device, when used as a suture passer, would, in some embodiments, be capable of passing one or more sutures, wires, loops, flexible metal jaw hooks, or any appropriate means of securing a tendon to make a rotator cuff repair. In some embodiments, a metal wire having, for example, a looped or diamond-shaped end may be threaded through the device (e.g. through an aperture, eyelet, groove, channel, etc.) and used to pull or thread one or more sutures back through the device.

The suture passer is preferably of substantially the same shape and cross-sectional size as the device described above. “Substantially the same shape”, as referred to herein, indicates that the suture passer is shaped so that its hooked portion is able to fit into (and its tip pass through) a bone channel formed by the awl described above. In some embodiments, the suture passer may also be of a smaller cross-sectional size than the awl.

The hooked portion has a selected radius of curvature of 9 to 15 mm, more preferably 10 to 14 mm, more preferably 11 to 13 mm most preferably about 12 mm. The hooked portion comprises 150 to 210 degrees, preferably 160 to 210 degrees, more preferably 170 to 210 degrees, more preferably still 180 to 210 degrees, even more preferably 190 to 210 degrees, or even more preferably still 195 to 205 degrees of arc. Preferably, it comprises about 199 degrees of arc. In one embodiment, the hooked portion comprises about 199 degrees of arc. It may have a curvature similar to a curved needle. The size of the hooked portion may depend on the age and size of the patient.

Preferably, the suture passer has a substantially round cross-sectional shape. However, it may also have a square, diamond-shaped or oval cross-sectional shape. Whatever the cross-sectional shape, a skilled person would appreciate that the suture passer is sized to fit into the curved bone tunnel formed by the awl.

In some embodiments, the shaft 210 and hooked portion 230 may be angled with respect to one another. In such embodiments, they may be angled 30 to 60, more preferably 40 to 50, or still more preferably about 45 degrees with respect to one another. The angle recapitulate the angle between a curved needle and a needle holder as used during surgery.

As seen in FIG. 2B and 2F, the tip 240 of suture passer 20 is preferably blunt. More preferably, it is a rounded point or round. However, the tip 240 may also be pointed or sharp, as may be seen in FIGS. 2A or 2G.

It is preferred that the hooked portion 230 of the suture passer be longer than the hooked bone-penetrating portion 130 of the awl to facilitate passage of the suture through the curved bone tunnel. Tip 240 may extend 0 to 6 mm beyond 180 degrees of arc. Preferably, tip 240 extends 2 to 6 mm beyond 180 degrees of arc. Even more preferable, tip 240 terminates about 4 mm beyond 180 degrees of arc.

The suture passer is configured to hold one or more sutures or metal wire during normal use.

By “hold” (as used throughout) is meant that the suture passer would retain a suture securely enough to allow it to be passed through a bone channel. In some embodiments, “hold” is intended to encompass receiving or passing a suture through a bone tunnel. In some embodiments, “hold” encompasses a suture being received or passed through a suture which itself has already been passed through or engaged with a bone tunnel.

By “configured to hold” is meant that the suture passer is capable of receiving, holding, or passing a suture. In some embodiments, it may encompass the device having one or more aperture, eyelet, opening, groove, hook, recess, or channel through which a suture may be threaded. The means of holding may also encompass a means for releasably securing a suture to part of the device. For example, adhesives, clips, or clasps may be used to secure a suture to part of the device.

Referring to FIG. 2C, the suture passer may define a channel 280 or cannula configured to receiving or hold one or more suture or metal wires. Preferably the channel 280 terminates at an opening 290 defined at or near the tip. FIG. 2H depicts one preferred example of a channel 280 exiting at an opening 290 defined by tip 240. Opening 290 may be round, but preferably it is oval. The second end of channel 280 may be at an opening defined by the proximal end 260 of the shaft 210. In other embodiments, the second end of channel 280 may be at an opening situated elsewhere along the shaft 210 or hooked portion 230. The channel may be of any size suited to receiving one or more sutures or metal wires which provides a suture passer of sufficient strength for normal use.

Like the opening 290, the channel 280 is preferably sized to receive and pass two or three heavy sutures, a tape, or a wire-loop.

Other means for holding or passing one or more suture or metal wire in or on the suture passer during use are also fully contemplated herein. For instance, one or more sutures or metal wires may be held in an accommodating groove extending along at least a portion of the shaft 210 or along the entire length of the shaft 210. The one or more sutures or metal wires may also be threaded through eyelets spaced along the shaft 210 of the suture passer. These means of retaining may also include fasteners for reversibly securing the one or more suture or metal wire to the suture passer during normal use. These means of securing may involve threading the suture or metal wire through an opening or eyelet situated at or near the tip 240.

Referring to FIG. 2B, hooked portion 230 may define an eyelet 270 configured to receive or hold one or more suture or metal wire. Preferably, the eyelet 270 is situated towards the tip 240. Preferably, as depicted, for example, in FIG. 2F and 2G, the eyelet 270 is oval, though it may also be round. Even more preferably, it is oval and sized about 1.25 mm×1.95 mm. However, an eyelet of any shape or size suited to receiving and passing one or more sutures or metal wires is fully contemplated herein.

As depicted in FIG. 2E, the eyelet 270 may be in communication with a channel 280 extending through at least part of the hooked portion 230 so that a suture may be passed therethrough. Preferably, the channel should be sized to receive two or three heavy sutures. The channel may pass through the length of the shaft 210 and terminate at an opening in the proximal end 260. However, it may also pass through a shorter portion of the shaft and to an opening defined elsewhere on the shaft.

The hooked end 230 of the shaft may taper towards the tip 240. The substantially straight portion 220 may taper towards the hooked end 230. The substantially straight portion may taper from 3 to 6.5 mm to 1.2 to 2.5 mm. Preferably, the substantially straight portion tapers from 6.25 mm to 2.5 mm.

The shaft 210 or handle 250 comprises a flat surface to allow percussion, for example, with a mallet. Preferably, the proximal end of the shaft 260 may be flat to allow percussion. In a preferred embodiment, depicted in FIG. 2D, the handle 250 may be of greater diameter than the shaft 210 and may be shaped and sized to facilitate gripping. It may be formed of a material, such as silicone, rubber, metal, or plastic, to facilitate gripping and may also be textured for the same purpose. Preferably, it is formed of silicone.

The suture passer may be formed of stainless steel, such as stainless steel 455 in H900 condition. It may have a hardness minimum of 49 HRC (Rockwell scale).

The suture passer may be supplied in one piece, or may be assembled from parts, which may correspond to one or more of the gripping portion, shaft, hooked portion, or tip. Owing to the simplicity of construction, the suture passer or a part thereof may be readily sterilized, reused, and, therefore, may be more “environmentally friendly”. However, in another embodiment, the suture passer or a part thereof may be disposable and/or formed of aluminum or plastic.

Method of Making an Arthroscopic Rotator Cuff Repair

In another aspect, there is provided a method of making an arthroscopic rotator cuff repair using the aforementioned apparatus or kit.

In some embodiments, the method comprise steps of
penetrating said greater tuberosity of a humerus with the above-described device to form a curved bone channel, said awl comprising a shaft having a substantially straight proximal portion and a hooked distal portion comprising a sharp tip for penetrating bone;
passing a suture through said curved bone tunnel; and
securing a tendon to said greater tuberosity with said suture.

By creating a bone tunnel, the use of foreign bodies (e.g. anchors), and the specific complications related to their use may be avoided. For example, by avoiding suture anchors, some embodiments may decrease significantly the cost of execution of the surgical procedure

In some embodiments, the step of passing the suture is performed using the above-described device which may also function as a suture passer.

In other embodiments, the step of passing the suture is carried out with a suture passer as described above.

In certain embodiments, the penetrating step is accomplished by percussing said awl, preferably with a mallet.

In some embodiments, the penetrating step comprises:
penetrating said greater tuberosity in a first location to form a first partial curved bone tunnel, and
penetrating said greater tuberosity at second location to form a second partial curved bone tunnel in communication with said first partial bone tunnel.

When the bone is relatively hard, one or both penetrating steps may involve initial penetration with a straight instrument. For example, an initial cortical perforation can initially before performed with a straight instruments, such as a straight punch or drill. The initial perforation may encompass approximately the first millimetre of bone.

It is preferred that said first and second partial curved bone tunnels together form a curved bone tunnel having a substantially continuous curvature. Owing to the shape of the bone tunnel (which reflects the shape of the hooked portion of the awl), in some embodiments, there is provided stronger bone tunnels in fragile or osteoporotic bone than straight, shallow tunnels (which may be weaker) or suture anchors (which tend to pull out) could provide. Further, the substantially continuous curvature facilitates passage of one or more sutures, since there are no corners to negotiate.

It is preferable that said first location be a superior aspect of said greater tuberosity and said second location (the “lateral portal”) be the lateral distal cortex of the humerus. Preferably, the second location may be 1 cm or more below the proximal tip of the greater tuberosity. More preferably, the second location is about 1 cm below the proximal tip of said greater tuberosity.

The penetrating step may involve placing the tip against said greater tuberosity at an angle of about 90 degrees and penetrating the bone by manually pressing the awl into the bone. The penetration may also be awled by percussion, for example, the awl may be placed with the tip at an angle which is less than 90 degrees so that, when the awl is percussed, a component of the percussive force is directed against the bone surface to penetrate it.

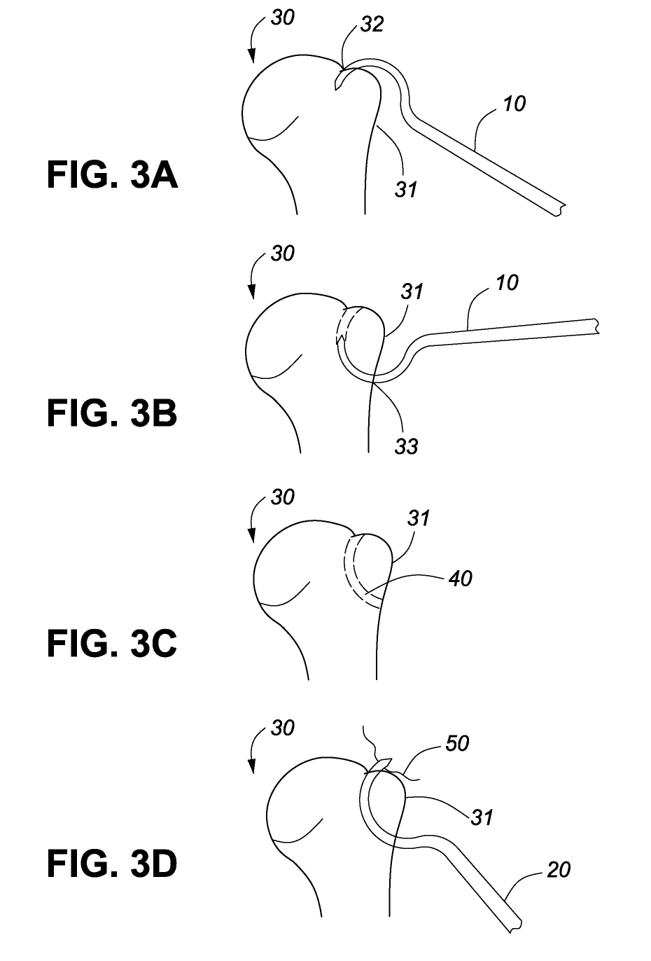

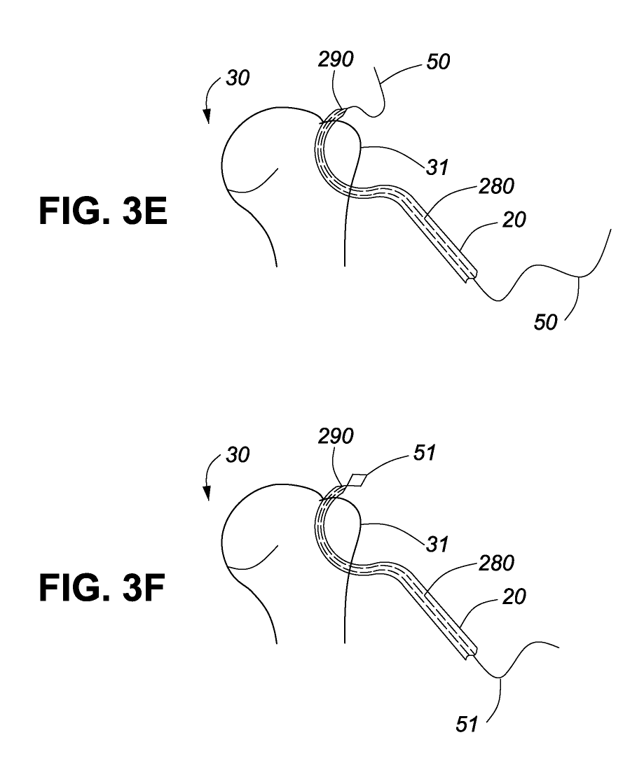

Referring to FIGS. 3A to 3D, in some embodiments, the method may comprise an arthroscopic visualisation and preparation of the humeral greater tuberosity, followed by an introduction of the awl by the lateral portal.

Referring to FIG. 3A, the superior aspect 32 of the tuberosity 31 of humerus 30 may be pierced by the awl 10 at the expected exit to form a first partial bone tunnel. Then, referring to FIG. 3B, the awl 10 may be placed at the lateral distal cortex 33 of the humerus 30 and percussive perforation may be gently made to form the second partial bone tunnel. In some embodiments, the awl may be placed 1 cm below the proximal tip of the tuberosity 31 for the second perforation. The tip of the awl may penetrate the bone in the direction of the first tunnel and be visualised when the two tunnels are joined. Referring to FIG. 3C, the awl may then be retracted from the patient to yield a complete bone tunnel 40 of substantially continuous curvature. Referring to FIG. 3D, the curved suture passer 20 loaded with one or more sutures 50 (which may, in some embodiments, be heavy sutures such as #2 nonresorbable sutures or tape) may engage the tunnel, for example, from the distal entry, and may transport a portion of the suture to the proximal exit. Once the sutures 50 are visualised at the proximal exit, they may be grasped and pulled. The suture may then be passed through the tendon, and an arthroscopic knot made to bring the tendon in contact with the bone and create the desired level and area of compression.

FIG. 3E depicts another embodiment in which suture 50 has been passed through the bone tunnel by a suture passer 20 comprising a channel 280 configured to receive and pass one or more sutures. A mechanism for pushing the suture forward through the channel of the awl (when used as a suture passer) or the suture passer may be provided. The mechanism may be mechanical, and for example, comprise one or two pulley wheels adapted to the shaft or the handle of the instrument. The mechanism could also comprise manual feeding, in some embodiments.

FIG. 3F depicts an embodiment in which suture passer 20 has a metal wire 51 having diamond-shaped head extending therethrough. This may be used to capture one or more suture, when may then be pulled back through the suture passer. The metal wire having a diamond-shaped head (which is merely an example) may also be used to pull the one or more sutures through a bone tunnel while the suture passer is engaged with a bone tunnel. A skilled person would appreciate that the suture pass could pass one or more sutures, wires (including metal wires), loops, or flexible metal jaw hooks, etc.

Due to the simplicity of the implements, some embodiments allow complete visualisation of the whole surgical procedure, in a strictly safe zone relatively far from the axillary nerve, thereby decreasing risks of iatrogenic nerve damage.

The above-described embodiments are intended to be examples only. Variations, alterations and modifications can be made to the particular embodiments described herein by those of skill in the art without departing from the scope, which is defined on the basis of the claims appended hereto.

What is claimed is:

1. A device for use in making an arthroscopic rotator cuff repair comprising:
a hooked bone-penetrating portion for creating a curved bone tunnel,
a shaft for transmitting force to said hooked bone-penetrating portion, and
a gripping portion for manipulating said device,
wherein said hooked bone-penetrating portion comprising a sharp tip for penetrating bone and further comprises 140 to 200 degrees of arc.

2. The device of claim 1 wherein said hooked bone-penetrating portion comprises 180 to 200 degrees of arc.

3. The device of claim 2 wherein said hooked bone-penetrating portion comprises a radius of curvature of about 12 mm.

4. The device of claim 1, wherein said device is configured to hold a suture, wire, loop, or flexible metal jaw hook.

5. The device of claim 4, wherein said device defines configured to hold said suture, wire, loop, or flexible metal jaw hook, said channel extending from a first opening defined in said hooked bone-penetrating portion to a second opening defined in said shaft.

6. The device of claim 4, wherein said device defines an eyelet configured to hold said suture, wire, loop, or flexible metal jaw hook, said eyelet being defined in said hooked bone-penetrating portion.

7. The device of claim 1, wherein said shaft and said hooked bone-penetrating portion are angled with respect to one another to recapitulate an angle between a curved surgical needle and a needle holder.

8. A suture passer for passing a suture, wire, loop, or flexible metal jaw hook through a curved bone tunnel, said suture passing comprising:
a hooked portion for passing through said bone tunnel,
a shaft coupled to said hooked portion, and
a gripping portion for manipulating said suture passer,
wherein said suture passer is configured to hold a suture, wire, loop, or flexible metal jaw hook, said hooked portion of said suture passer comprising 140 to 210 degrees of arc.

9. The suture passer of claim 8, wherein said curved bone tunnel is formed by the device of claim 1.

10. The suture passer of claim 8, wherein said suture passer is of substantially the same shape and of a cross-sectional thickness which is less than or equal to said device of claim 1.

11. The suture passer of claim 8 wherein said hooked portion of said suture passer comprises 190 to 210 degrees of arc.

12. The suture passer of claim 8, wherein said hooked bone-penetrating portion of said device and said hooked portion of said suture passer each have a radius of curvature of about 12 mm.

13. The suture passer of claim 8, wherein said suture passer defines a channel configured to hold said suture, wire, loop, or flexible metal jaw hook, said channel extending from a first opening defined in said hooked portion to a second opening defined in said shaft.

14. The suture passer of claim 8, wherein said suture passer defines an eyelet configured to hold said suture, wire, loop, or flexible metal jaw hook, said eyelet being defined in said hooked bone-penetrating portion.

15. The suture passer of claim 9, wherein said hooked portion of said suture passer is longer than said hooked bond-penetrating portion of said device.

16. A kit comprising the device of claim 1 together with a suture passer for passing a suture, wire, loop, or flexible metal jaw hook through a bone tunnel formed by the device of claim 1, said suture passing comprising:
a hooked portion for passing through said bone tunnel,
a shaft coupled to said hooked portion, and
a gripping portion for manipulating said suture passer,
wherein said suture passer is configured to hold a suture, wire, loop, or flexible metal jaw hook, said hooked portion of said suture passer comprising 140 to 210 degrees of arc.

17. The kit of claim 16, wherein said hooked bone-penetrating portion of said device and said hooked portion of said suture passer each have a radius of curvature of about 12 mm.

18. The kit of claim 16, wherein said hooked portion of said suture passer comprises 190 to 210 degrees of arc.

19. The kit of claim 16, wherein said suture passer defines a channel configured to hold said suture, wire, loop, or flexible metal jaw hook, said channel extending from a first opening defined in said hooked portion to a second opening defined in said shaft.

20. The kit of claim 16, wherein said suture passer defines an eyelet configured to hold said suture, wire, loop, or flexible metal jaw hook, said eyelet being defined in said hooked bone-penetrating portion.

21. The kit of claim 16, wherein said hooked portion of said suture passer is longer than said hooked bone-penetrating portion of said device.

22. A method of making an arthroscopic rotator cuff repair comprising the steps of
penetrating the greater tuberosity of a humerus with the device of claim 1 to form a curved bone channel;
passing a suture, wire, loop, or flexible metal jaw hook through said curved bone tunnel; and
securing a tendon to said greater tuberosity with said suture, wire, loop, or flexible metal jaw hook to make said rotator cuff repair.

23. The method of claim 22, wherein said hooked bone-penetrating portion comprises 180 to 200 degrees of arc.

24. The method of claim 22, wherein said hooked bone-penetrating portion comprises a radius of curvature of about 12 mm.

26. The method of claim 22, wherein passing is accomplished with said device, said device being configured to hold a suture, wire, loop, or flexible metal jaw hook.

27. The method of claim 26, wherein said device defines a channel configured to hold said suture, wire, loop, or flexible metal jaw hook, said channel extending from a first opening defined in said hooked bone-penetrating portion to a second opening defined in said shaft.

28. The method of claim 26, wherein said device defines an eyelet configured to hold said suture, wire, loop, or flexible metal jaw hook, said eyelet being defined in said hooked bone-penetrating portion.

29. The method of claim 22, wherein passing is accomplished with a suture passer comprising:
a hooked portion for passing through said bone tunnel,
a shaft coupled to said hooked portion, and
a gripping portion for manipulating said suture passer,
wherein said suture passer is configured to hold a suture, wire, loop, or flexible metal jaw hook, said hooked portion of said suture passer comprising 140 to 210 degrees of arc.

30. The method of claim 29, wherein said hooked portion of said suture passer comprises 190 to 210 degrees of arc.

31. The method of claim 29, wherein said hooked bone-penetrating portion of said device and said hooked portion of said suture passer each have a radius of curvature of about 12 mm.

32. The method of claim 29, wherein said suture passer defines a channel configured to hold said suture, wire, loop, or flexible metal jaw hook, said channel extending from a first opening defined in said hooked portion to a second opening defined in said shaft.

33. The method of claim 29, wherein said suture passer defines an eyelet configured to hold said suture, wire, loop, or flexible metal jaw hook, said eyelet being defined in said hooked bone-penetrating portion.

34. The method of claim 22, wherein penetrating comprises:
penetrating said greater tuberosity at a first location to form a first partial curved bone tunnel, and
penetrating said greater tuberosity at a second location to form a second partial curved bone tunnel in communication with said first partial bone tunnel, said first and second partial curved bone tunnels together having a substantially continuous curvature.

35. The method of claim 22, wherein prior to penetrating, an initial perforation is made into said greater tuberosity with a straight instrument.
